(19) United States
(12) Patent Application Publication
(10) Pub. No.: US 2011/0196488 A1
(43) Pub. Date: Aug. 11, 2011

(54) DEGRADATION RESISTANT IMPLANTABLE MATERIALS AND METHODS

(75) Inventors: Alexei Goraltchouk, Santa Barbara, CA (US); Jordan M. Thompson, Scotts Valley, CA (US); Miriam M. Abiad, Costa Mesa, CA (US); Kevin A. Ma, Scotts Valley, CA (US)

(73) Assignee: Allergan, Inc., Irvine, CA (US)

(21) Appl. No.: 13/015,309

(22) Filed: Jan. 27, 2011

Related U.S. Application Data
(60) Provisional application No. 61/301,104, filed on Feb. 3, 2010.
(60) Provisional application No. 61/375,338, filed on Aug. 20, 2010.

(51) Int. Cl. A61F 2/12; B32B 5/18; B32B 27/08; B05D 7/00; B05D 1/38; B05D 3/00; B05D 1/36

(52) U.S. Cl. 623/8; 428/322.7; 427/2.24

(57) ABSTRACT

Methods are provided for making materials suitable for implantation in a mammal. The methods include the steps of providing a base material having a desirable surface topography, such as a polyurethane foam, contacting the base member with a silicone-based fluid material to form a coating, and allowing the coating to set to form a silicone-based structure suitable for implantation in a mammal. The base material may be removed from the coating.

### RELATED APPLICATIONS

This application claims priority to U.S. Provisional Patent Application No. 61/301,104, filed on Feb. 3, 2010 and U.S. Provisional Patent Application No. 61/375,338 filed on Aug. 20, 2010, the disclosure of each of these applications incorporated herein in its entirety by this reference.

The present invention generally relates to medical implants and more specifically relates to foam-like materials suitable for implantation in a mammal.

Prostheses or implants for augmentation and/or reconstruction of the human body are well known. Capsular contracture is a complication associated with surgical implantation of prostheses, particularly with soft implants, and even more particularly, though certainly not exclusively, with fluid-filled breast implants.

Capsular contracture is believed to be a result of the immune system response to the presence of a foreign material in the body. A normal response of the body to the presence of a newly implanted object, for example a breast implant, is to form a capsule of tissue, primarily collagen fibers, around the implant. Capsular contracture occurs when the capsule begins to contract and squeeze the implant. This contracture can be discomforting or even extremely painful, and can cause distortion of the appearance of the augmented or reconstructed breast. The exact cause of contracture is not known. However, some factors may include bacterial contamination of the implant prior to placement, submuscular versus subglandular placement, and smooth surface implants versus textured surface implants, and bleeding or trauma to the area.

Surface texturing has been shown to reduce capsular contracture when compared to what are known as “smooth” surface implants.

There is still a need for a more optimal surface textured implant that further reduces the potential for capsular contracture. The present invention addressed this need.

### SUMMARY OF THE INVENTION

Accordingly, the present invention provides a method of making a material suitable for implantation in a mammal. The method generally comprises the steps of providing a base member including a porous surface defined by interconnected pores and contacting the base member with a silicone-based fluid material in a manner to cause the fluid material to enter the pores. In one embodiment, a vacuum is applied to the base member to draw the fluid material into and/or through the pores. The method may comprise the steps of removing excess fluid material from the base member to obtain a coating of the fluid material on the porous surface, and allowing the coating to set to form a silicone-based structure suitable for implantation in a mammal. The removal process can be obtained using an airknife to blow away the excess material, and/or squeezing out the excess material, and/or using suction to remove the excess material. The silicone-based structure includes a porous surface, having interconnected cells, the porous surface substantially identically conforming to the porous surface of the base member.

In one aspect of the invention, the base material is a material which can be degraded or otherwise removed from within the coating without substantially affecting the coating structure. In some embodiments, the base material is a substantially biodegradable material. The base material may be polyurethane, for example, polyurethane foam. Alternatively, the base member is melamine, for example, melamine foam. Other base member materials are also contemplated and include, for example, foams made from polyethylene, polyethylene vinyl acetate, polystyrene, polyvinyl alcohol, or generally a polyolefin, polyester, polyether, polyamide, polysaccharide, a material which contains aromatic or aliphatic structures in the backbone, as functionalities, crosslinkers or pendant groups, or a copolymer, terpolymer or quarternaly polymer thereof. Alternatively the material may be a composite of one or more aforementioned materials. In another embodiment of the invention the base material can be a metal, for example a metal foam, a ceramic, or a composite material.

The silicone-based fluid material may comprise a dispersion, for example, a silicone dispersion, solution, emulsion or mixture. The silicone-based fluid material may be a solution of a room temperature vulcanizing (RTV) or a high temperature vulcanizing (HTV) silicone from about 0.1-95 wt %, for example, about 1-40 wt %, for example, about 30 wt %. In an exemplary embodiment, the silicone-based fluid material is a high temperature vulcanizing (HTV) platinum-cured silicone dispersion in xylene.

In another aspect of the invention, the base member, or at least a portion thereof, is removed from the silicone-based structure. In one embodiment, substantially all of the base material is removed, such that a product is obtained which comprises or consists of material that is substantially entirely pure silicone, for example, a porous, cellular silicone foam. The step of removing may comprise, for example, contacting the base member with a solution capable of dissolving the base member. For example, in an embodiment of the invention in which the base member is polyurethane foam, the step of removing may comprise contacting the base member with a hydrogen peroxide solution. In other embodiments of the invention, the base material may be degraded by exposure to UV light, heat, oxidative agents, a base such as sodium hydroxide, or an acid such as phosphoric acid or a combination thereof. The material may be exhaustively removed further by a secondary process such as solvent leach or vacuum.

In another aspect of the invention, a material suitable for implantation in a mammal is provided. The material comprises a porous, cellular member comprising a silicone-based structure. The silicone-based structure has a topography, for example, a pore size, shape and interconnectivity, substantially identical to that of a polyurethane foam. This material may be made by the processes in accordance with methods of the invention, as described herein.

In yet another aspect of the invention, a method of making a material suitable for implantation in a mammal is provided which generally comprises providing a base member comprising a degradable foam and including a porous surface defined by interconnected pores, and coating the base member with a substantially non-biodegradable polymeric material to obtain a substantially non-biodegradable polymeric structure suitable for implantation in a mammal. More specifically, the method includes contacting the base member with a fluid precursor of the substantially non-biodegradable polymeric material in a manner to cause the fluid precursor to enter the pores, removing excess fluid precursor material to obtain a coating of the fluid precursor on the base member, and allowing the coating to set to form the substantially non-biodegradable polymeric structure. The resulting structure includes a porous surface substantially identically conforming to the porous surface of the base member.

In yet another aspect of the invention, a method is provided which generally comprises providing a base member including a porous surface defined by interconnected pores, contacting the base member with a first material, allowing the first material to set to form a first material coating on the base member, contacting the first material coating with a second material different from the first material and allowing the second material to set to form a layered polymeric structure suitable for implantation in a mammal. The resulting layered polymeric structure includes a porous surface substantially identically conforming to the porous surface of the base member. In an exemplary embodiment, the first material is a fluorinated polyolefin material and the second material is a silicone dispersion.

In yet further aspects of the invention, methods for augmenting or reconstructing a human breast are provided, wherein the methods comprise implanting, in a human breast, a material made by the methods described herein.

Each and every feature described herein, and each and every combination of two or more of such features, is included within the scope of the present invention provided that the features included in such a combination are not mutually inconsistent.

### BRIEF DESCRIPTION OF THE DRAWINGS

The present invention may be more clearly understood and certain aspects and advantages thereof better appreciated with reference to the following Detailed Description when considered with the accompanying Drawings of which:

FIG. 1 is an SEM micrograph of a implantable material made in accordance with a method of the invention; and.

FIG. 2 is an SEM micrograph of a melamine foam which can be used as a base member in accordance with a method of the invention.

FIGS. 3-9 are images of other materials that can be useful as base materials in accordance with different embodiments of the invention.

### DETAILED DESCRIPTION

The present invention generally pertains to implantable materials and methods of forming implantable materials. The materials may be used as coverings or outer layers for implants, such as breast implants, and are designed to at least reduce the risk of capsular contracture.

In one aspect of the invention, methods are provided for making an implantable material that is substantially biologically inert and/or substantially non-biodegradable, which has a structure, for example, a microstructure, similar or substantially identical to that of a foam of a different material. The different material may be, or may not be, a biologically inert or non-biodegradable material.

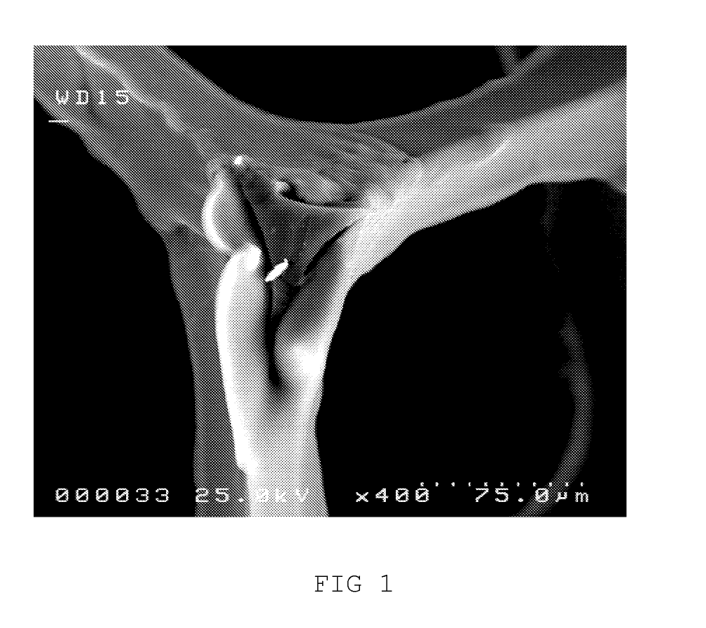

In a specific embodiment, the implantable materials are substantially entirely comprised of silicone yet have the topographical structure of a polyurethane foam. For example, a material in accordance with one embodiment is a flexible, soft, silicone-based foam having substantially the same or substantially identical geometry and tissue disorganization potential of a polyurethane foam, but with the chemical inertness and biocompatibility of a silicone. FIG. 1 is an SEM image of a polyurethane foam strut coated with silicone elastomer, in accordance with an embodiment of the invention.

For example, a method for making an implantable material substantially entirely comprised of silicone, in accordance with one embodiment of the invention, generally comprises the steps of providing a polyurethane base member including a porous surface defined by interconnected pores, contacting the base member with a silicone-based fluid material in a manner to cause the fluid material to enter the pores. A vacuum may be applied to the base material in order to facilitate the contacting step. Excess fluid material may be removed from the base member to obtain a coating of the fluid material on the porous surface. The silicone-based coating is allowed to set to form a silicone-based structure. The coating steps may be repeated once, twice, three or more times, for example, up to 1000 times, until a desired thickness and/or final foam density is achieved. The underlying polyurethane material may be removed from the coating structure. For example, the polyurethane is contacted with a dissolvent, dimethyl sulfoxide, or a degradant such as hydrogen peroxide or hydrochloric acid, followed by a dissolvent such as dimethyl sulfoxide of dimethyl formamide or acetone. The resulting silicone-based material is flexible and biocompatible and includes a porous surface substantially identically conforming to the porous surface of a polyurethane foam.

It is to be appreciated that for a base material other than polyurethane, said base material can be removed by a solvent or other means, known to those of skill in the art, suitable for removing the base material from the coating without substantially altering or affecting the coating structure.

The base material may have a pore size of about 100-1000 μm (RSD, i.e. relative standard deviation, of about 0.01-100%); an interconnection size of about 30-700 μm (RSD of 0.01-100%); interconnections per pore of about 2-20(RSD of 0.01-50%); and an average pore to interconnection size ratio of about 3-99%.

In some embodiments, the base material has a pore size of about 300-700 μm (RSD of 1-40%); an interconnection size of about 100-300 μm (RSD of 1-40%); interconnections per pore of about 3-10 (RSD of 1-25%) and an average pore to interconnection size ratio of about 10-99%.

In an exemplary embodiment, the base member comprises a material, for example, polyurethane or other suitable material, having a pore size of 472+/−61 μm (RSD=13%), interconnection size: 206+/−60 μm (RSD=29%), interconnections per pore: 9.6+/−1.8 (RSD=19%), Pore to interconnection size ratio of 44%.

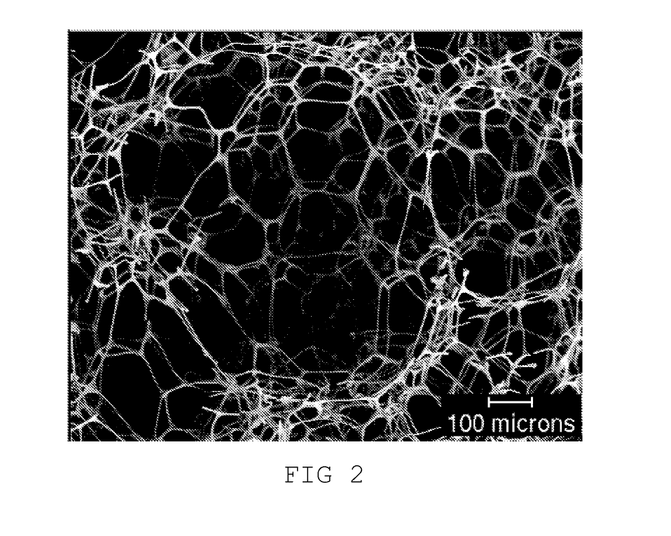

The base member may comprise any suitable porous material having the desired surface structure. Alternative to polyurethane, the base member may comprise melamine, for example, melamine foam. FIG. 2 is an SEM micrograph of a melamine foam having a topography defined by highly interconnected, open pores. Other base member materials useful in the methods of the invention are also contemplated and include, for example, polyethylene foam, Styrofoam, or general polyolefin foams, polysaccharide foams, polyamide foams, polyacrylate foams, metal and ceramic foams.

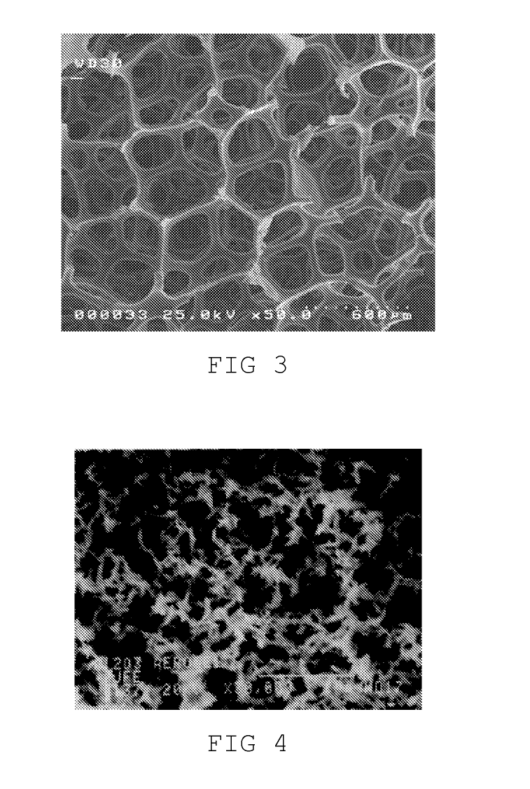

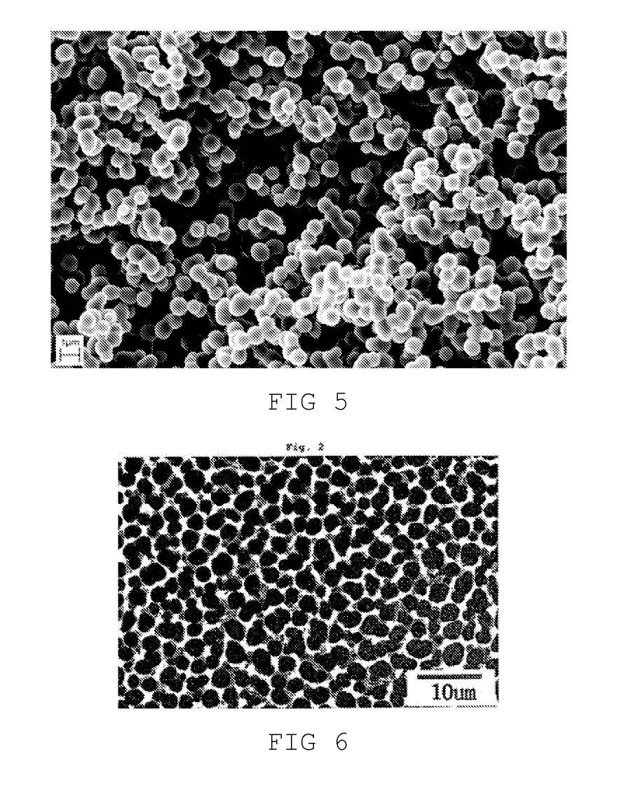

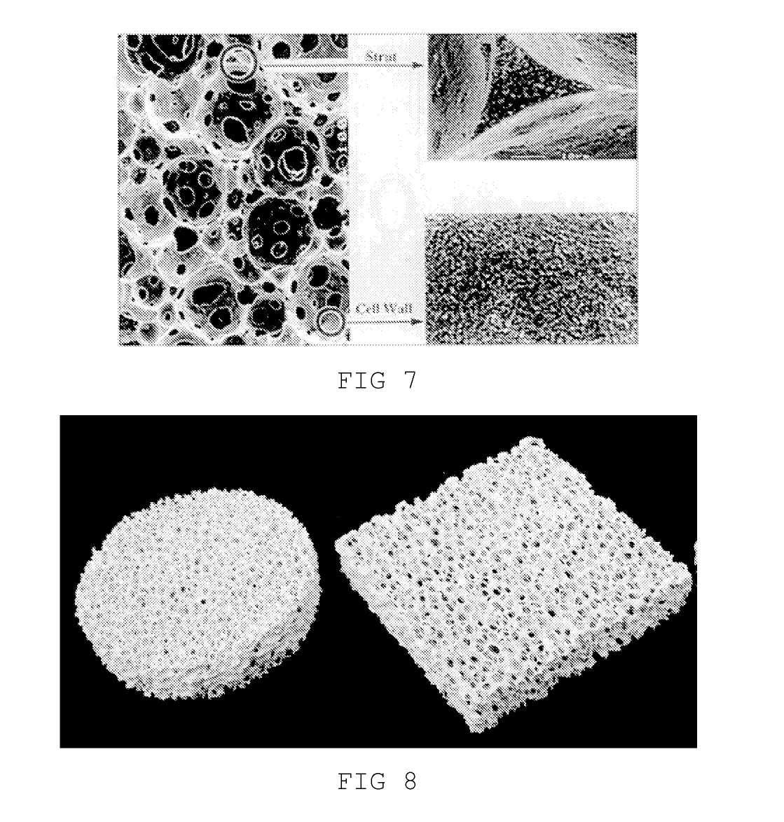

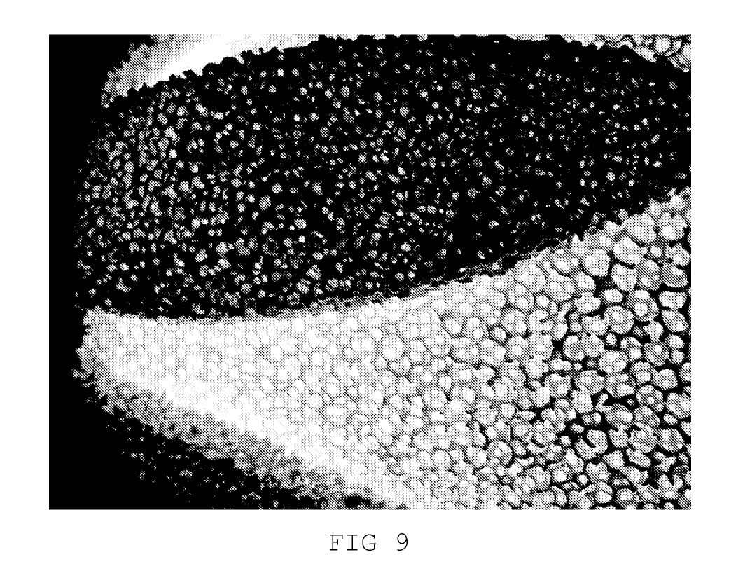

Porous surfaces of base member materials useful in accordance with various embodiments of the invention are shown in FIGS. 3-10. More specifically, FIG. 3 is a SEM image of a polyurethane foam base; FIG. 4 is an alumina aerogel foam; FIG. 5 is another aerogel, for example, silica aerogel foam; FIG. 6 is a silica foam; FIG. 7 is a HiP foam; FIG. 8 is a magnesium ceramic foam; and FIG. 9 is another ceramic foam.

In an exemplary embodiment, the silicone-based fluid material may comprise a dispersion, for example, a silicone dispersion. The silicone-based fluid material may be a room temperature vulcanizing (RTV) or a high temperature vulcanizing (HTV) silicone. In an exemplary embodiment, the silicone-based fluid material is a high temperature vulcanizing (HTV) platinum-cured silicone dispersion in xylene or chloroform.

Alternatives to silicone-based polymers are also contemplated. For example, any implantable material that can be cured by crosslinking, thermoplastics that set by change in temperature, material that set by removal of solvents or any elastomer that cures or sets by any known mechanism, can be used. It is further contemplated that other implantable materials useful in accordance with the invention include suitable metals or ceramics.

The type of polymeric fluid material forming the coating on the base member, the total dissolved solids of the coating material, the method of removing the excess fluid, the carrier solvent, the method of applying the coating solution, the temperature of the solution, can be varied in accordance with different embodiments of the invention.

In some embodiments, base material is coated with multiple layers of different materials. For example, a first coating material may comprise a barrier layer of a material capable of reducing or preventing diffusion of chemical substances from the base material, and a second coating applied on the first coating may comprise a silicone-based material. Other coating materials may be selected to achieve various characteristics of the final product, such as materials to strengthen the foam, prevent chemical degradation, and/or change surface properties.

In yet another aspect of the invention, a method of making a material suitable for implantation in a mammal is provided which generally comprises providing a base member comprising a degradable foam and including a porous surface defined by interconnected pores, and coating the base member with a substantially non-biodegradable polymeric material to obtain a substantially non-biodegradable composite structure suitable for implantation in a mammal. For example, the base member may comprise a polyurethane foam. The substantially non-biodegradable polymeric material can be any suitable biocompatible polymer and may be selected from a list of highly impermeable systems such as fluorinated polymers to prevent diffusion of chemical entities which may facilitate the degradation of polyurethane. Alternatively, the fluorinated polymer can be applied as a base layer, prior to a final application of the silicone, to act as a barrier layer.

For example, in one embodiment of the invention, a method of making a textured material, for example, but not limited a porous material suitable for implantation in a mammal, is provided wherein the method comprises the steps of providing a base material comprising polyurethane foam having a surface defined by interconnected pores and contacting the base material with a fluorinated polymeric material in a manner to cause the fluorinated polymeric material to enter the pores. A vacuum and/or air blower or airknife may be applied as described elsewhere herein to facilitate intimate and uniform contact between the materials. The composite material thus formed has a fluorinated polymer surface defined by interconnected pores that are substantially identical to those of the polyurethane foam surface. In one embodiment, the fluorinated polymeric material is a fluorinated polyolefin. In another embodiment, the method may further comprise the step of contacting the fluorinated polymeric surface with a silicone-based material in a manner to form a silicone-based coating on the fluorinated polymeric surface. A textured prosthesis may be assembled by applying or attaching this composite material to a surface of an implantable device, for example, a breast prosthesis.

In another embodiment of this invention, the base member of a preferred geometry, that is not dissolvable (for example, a crosslinked polymer having a porous surface) may be coated by a robust but dissolvable material, such as, for example, a foam material selected from the group of materials consisting of polystyrene, polyethylene-co-vinyl acetate, and poly(styrene-co-butadiene-co-styrene). The base member, e.g. the non-dissolvable foam, can then be removed from the dissolvable material coating, for example, degraded by relatively aggressive means, for example, by acid digestion in 37% HCl, leaving the robust but dissolvable material behind. An implantable material of interest, for example, a silicone-based fluid material, is deposited on the robust but dissolvable foam, for example, using the methods described elsewhere herein. The silicone-based fluid material may be in the form of a dispersion having a solvent system that does not dissolve the robust polymer. The silicone is allowed to set or cure, and the robust material is then dissolved out by means which does not affect the material of interest (e.g. silicone), for example, by dissolution in acetone in the case of polystyrene. In this case, the material of interest is not subjected to aggressive conditions used to dissolve the original foam.

### Example 1

A polyurethane open celled foam is coated according to the current invention using a solution of Silicone HTV 30% w/v, by either dipping the polyurethane foam in the solution, casting the solution on a sheet of polyurethane or spraying the solution in excess over the sheet of polyurethane. The excess solution is removed by squeezing out the foam, or by vacuum which is applied through a Buchner funnel at the bottom of the foam (in the case of casting the solution over the foam) or by blowing air over the foam as in the case of an air-knife, or in combination of any of the aforementioned. The foam is then devolitilized in vacuum or by application of mild heat in the case of HTV, such that the solvent is removed, but the HTV is not cured. This can be achieved in the application of the air current during the previous step (the air may or may not be heated). Finally the coated foam is cured and the coating layer is affixed unto the foam. The process may be repeated from 1 to about 1000 times (more specifically 1, 4 times) to achieve various builds (final pore densities). The polyurethane is completely removed from the center of the structure by digestion in hydrogen peroxide/water solution with or without the presence of metal ions and with or without heating. Alternatively the polyurethane foam can be degraded out by 37% HCl digestion for 1-5 minutes, with vigorous agitation and air removal to facilitate the uniform digestion of the polyurethane, and a subsequent DMSO wash to remove the remnant degradants which are not soluble in the 37% HCl. The degradation/leaching steps can be repeated 1-20 times to achieve various levels of purity. The resulting material is a substantially pure silicone foam useful as a surgical implant.

### Example 2

A sheet polyurethane open celled foam (20×20 cm) is placed in a container the bottom of which is a fine grate. Vacuum is applied to the bottom of the grate to pull air through the top of the foam into the foam and finally through the grate and out. A solution of about 20% HTV (platinum cured) in chloroform is cast over the foam and pulled through the foam by the vacuum, a jet of air is applied to the foam through an air-knife to remove any remaining solution droplets that are trapped in the foam to clean out the pores. The foam is then devolitized in vacuum at about room temperature for 2 hours. The devolitized foam is finally cured at 120° C. for 1 hour. The process is repeated 3 times. The resulting foam is an open celled polyurethane base foam, conformally coated by an approximately 50 μm layer of silicone.

### Example 3

A implantable material is produced substantially in accordance with Example 1, except that instead of a polyurethane foam, a melamine foam is used as the base member. In addition, the base material is not removed from the silicone foam. The resulting implantable material comprises a highly porous, open celled structure having a melamine base and a silicone overcoat.

### Example 4

The silicone foam of Example 1 is produced as a flexible sheet. The sheet is cut and laminated to form a front surface of a breast implant. The front surface of the breast implant has a surface texture substantially identical to a surface texture of a polyurethane foam, but is substantially pure silicone.

### Example 5

A sheet of polyurethane open celled foam base material (20×20 cm) is placed in a container the bottom of which is a fine grate. Vacuum is applied to the bottom of the grate to pull air through the top of the foam into the foam and finally through the grate and out. A solution of MED-4850, a high durometer silicone, is cast over the foam and pulled through the foam by the vacuum, a jet of air is applied to the foam through an air-knife to remove any remaining solution droplets that are trapped in the foam to clean out the pores. The foam is then devolitized in vacuum at about room temperature for 2 hours and cured at 120° C. for 1 hour.

Then, a second coating is applied by casting a solution of MED-4830, a lower durometer silicone, over the cured first coating. The solution is pulled through the foam by the vacuum, a jet of air is applied to the foam through an air-knife to remove any remaining solution droplets that are trapped in the foam to clean out the pores. The foam is then devolitized in vacuum at about room temperature for 2 hours and cured at 120° C. for 1 hour.

Then, a third coating is applied by casting a solution of MED-4815, an even lower durometer silicone, over the cured second coating. The solution is pulled through the foam by the vacuum, a jet of air is applied to the foam through an air-knife to remove any remaining solution droplets that are trapped in the foam to clean out the pores. The foam is then devolitized in vacuum at about room temperature for 2 hours and cured at 120° C. for 1 hour.

Then, a fourth final coating is applied by casting a solution of MED-4801, the lowest durometer silicone used, over the cured third coating. The solution is pulled through the foam by the vacuum, a jet of air is applied to the foam through an air-knife to remove any remaining solution droplets that are trapped in the foam to clean out the pores. The foam is then devolitized in vacuum at about room temperature for 2 hours and cured at 120° C. for 1 hour.

The resulting material is an open celled polyurethane base foam, conformably coated by an approximately 200 μm layer of decreasing durometer silicone. The polyurethane base material can be optionally removed from the composite member. Other composite materials can be similarly made.

### Example 6

A sheet of polyurethane open celled foam base material (20×20 cm) is placed in a container the bottom of which is a fine grate. Vacuum is applied to the bottom of the grate to pull air through the top of the foam into the foam and finally through the grate and out. An aqueous dispersion of fluorinated polyolefin (e.g. HYPOD™ Polyolefin Dispersions available from DOW Chemical Company) is cast over the foam and pulled through the foam by the vacuum. A jet of air is applied to the foam through an air-knife to remove any remaining solution droplets that are trapped in the foam and to clean out the pores. The fluorinated polyolefin coated foam is then heated at a sufficient temperature to allow the water in the aqueous dispersion to evaporate and the coating to melt. The fluorinated polyolefin coating is a uniform, fine film coating on the surfaces of the polyurethane foam. This coated polyurethane foam can then be bonded with a suitable, biocompatible adhesive to a smooth shell breast prosthesis which can then be implanted in a patient. The prosthesis will have the desirable characteristics of a polyurethane covered implant, that is, for example, the capsular tissue disorganization potential of polyurethane foam, but with the reduced chance of degradation of the polyurethane foam into the body.

### Example 7

A fluorinated polyolefin-coated polyurethane foam material is made as described in Example 6. However, before the material is bonded to a smooth shell breast prosthesis, a silicone coating is applied to the fluorinate polyolefin coating by casting a solution of MED-4830 over the fluorinate polyolefin coating. The silicone solution is pulled through the foam by the vacuum, and a jet of air is applied to the foam through an air-knife to remove any remaining solution droplets that are trapped in the foam to clean out the pores. The foam is then devolitized in vacuum at about room temperature for 2 hours and cured at 120° C. for 1 hour. The coated polyurethane foam is then bonded with a suitable, biocompatible adhesive to a smooth shell breast prosthesis.

While this invention has been described with respect to various specific examples and embodiments, it is to be understood that the invention is not limited thereto and that it can be variously practiced within the scope of the invention.

What is claimed is:

1. A method of making a material suitable for implantation in a mammal, the method comprising:
providing a base member including a porous surface defined by interconnected pores;
contacting the base member with a silicone-based fluid material in a manner to cause the fluid material to enter the pores;
removing excess fluid material from the base member to obtain a coating of the fluid material on the porous surface; and
allowing the coating to set to form a silicone-based structure suitable for implantation in a mammal, the silicone-based structure including a porous surface substantially identically conforming to the porous surface of the base member.

2. The method of claim 1 further comprising the step of applying a vacuum to the base member to draw the fluid material into the pores.

3. The method of claim 1 further comprising removing at least a portion of the base member from the silicone-based structure.

4. The method of claim 1 wherein the base member is substantially polyurethane.

5. The method of claim 1 wherein the base member is substantially melamine.

6. The method of claim 1 wherein the coating has a thickness of between about 10 microns and about 100 microns.

7. The method of claim 1 further comprising the step of removing substantially all of the base member from the coating after the step of allowing the coating to set.

8. The method of claim 7 wherein the step of removing comprises contacting the base member with a solution capable of dissolving the base member.

9. A method of making a material suitable for implantation in a mammal, the method comprising:
providing a base member comprising a biodegradable foam and including a porous surface defined by interconnected pores;
contacting the base member with a fluid precursor of a substantially non-biodegradable polymeric material in a manner to cause the fluid precursor to enter the pores;
removing excess fluid precursor from the base member to obtain a coating of the fluid precursor on the porous surface; and
allowing the coating to set to form a substantially non-biodegradable polymeric structure suitable for implantation in a mammal, the substantially non-biodegradable polymeric structure including a porous surface substantially identically conforming to the porous surface of the base member.

10. The method of claim 9 wherein the biodegradable foam is a polyurethane foam.

11. The method of claim 9 further comprising the step of removing at least a portion of the base member from the substantially non-biodegradable polymeric structure.

12. The method of claim 9 wherein the substantially non-biodegradable polymeric structure is substantially entirely silicone.

13. A method of making a material suitable for implantation in a mammal, the method comprising:
providing a base member made of a biodegradable foam and including a porous surface defined by interconnected pores;
contacting the base member with a fluorinated polyolefin material in a manner to cause the fluorinated polyolefin material to enter the pores;
allowing the fluorinated polyolefin material to set to form a fluorinated polyolefin coating on the base member;
contacting the fluorinated polyolefin coating with a silicone-based fluid material;
allowing the silicone-based fluid material to set to form a layered polymeric structure suitable for implantation in a mammal, the layered polymeric structure including a porous surface substantially identically conforming to the porous surface of the base member.

14. A method of making a material suitable for implantation in a mammal, the method comprising:
providing a polymeric base member having a surface defined by a geometry including interconnected pores;
forming a first coating on the surface of the base member material, the first coating being selected from the group of materials consisting of polystyrene, polyethylene-co-vinyl acetate, and poly(styrene-co-butadiene-co-styrene);
removing the polymeric base member by contacting the base material with a material that will cause the base member to be removed from the first coating without causing degradation of the coating;
applying a silicone-based fluid material to the first coating having the polymeric base member removed therefrom;
curing the silicone-based fluid material to form a silicone-based coating on the first coating; and
removing the first coating from the silicone coating.

15. The method of claim 14 wherein the step of removing the first coating comprises contacting the first coating with a material that dissolves the first coating without substantially affecting the silicone coating.

16. A method of making a material suitable for implantation in a mammal, the method comprising:
providing a base material comprising polyurethane foam having a surface defined by interconnected pores;
contacting the base material with a fluorinated polymeric material in a manner to cause the fluorinated polymeric material to enter the pores and form a fluorinated polymeric coating on the base material thereby forming a biocompatible, substantially non-biodegradable composite material.

17. The method of claim 16 wherein the fluorinated polymeric material is a fluorinated polyolefin.

18. The method of claim 16 further comprising the step of applying a silicone-based material to the fluorinated polymeric coating in a manner to form a conformal silicone-based coating on the fluorinated polymeric coating.

19. The method of claim 16 further comprising the step of applying vacuum to the base material during the step of contacting.

20. A composite material made by the method of claim 16.

21. A method for augmenting or reconstructing a human breast comprising the steps of:
implanting the material made by the method of claim 1 in a human breast.
